(19) United States
(12) Patent Application Publication
(10) Pub. No.: US 2026/0216461 A1
(43) Pub. Date: Jul. 30, 2026

(54) NECK-SUPPORTED MEDICAL GAS DELIVERY

(71) Applicant: Sleep Easy Technology LLC, South Bend, IN (US)

(72) Inventors: Anthony Esplin, South Bend, IN (US); Aaron Esplin, South Bend, IN (US)

(21) Appl. No.: 19/454,176

(22) Filed: Jan. 20, 2026

Related U.S. Application Data
(63) Continuation of application No. PCT/US24/38911, filed on Jul. 19, 2024.
(60) Provisional application No. 63/527,746, filed on Jul. 19, 2023.

(51) Int. Cl. A61M 16/06; A61M 16/08

(52) U.S. Cl. CPC A61M 16/0683; A61M 16/0875; A61M 2202/0007; A61M 2202/02; A61M 2209/088; A61M 2210/0606; A61M 2210/0618; A61M 2210/0625

(57) ABSTRACT

A gas delivery system that delivers gases that are potentially medical in nature includes a base positionable adjacent to (e.g., around, next to, etc.) an individual's neck and a diffuser carried by the base to deliver a medical gas to the individual's face (e.g., the individual's nose, the individual's nose and mouth, etc.). Methods for delivering medical gases are also disclosed.

### CROSS-REFERENCE TO RELATED APPLICATIONS

This application is a continuation of International Application Number PCT/US2024/038911, filed on Jul. 19, 2024 and titled NECK-SUPPORTED MEDICAL GAS DELIVERY (“the '911 PCT Application”). The '911 PCT Application includes a claim for priority to the Jul. 19, 2023 filing date of U.S. Provisional Patent Application No. 63/527,746, titled NECK-SUPPORTED MEDICAL GAS DELIVERY (“the '746 Provisional Application”). The entire disclosures of the '746 Provisional Application and the '911 PCT Application are hereby incorporated herein.

### TECHNICAL FIELD

This disclosure relates to devices for delivering gases that are potentially medical in nature (hereinafter “medical gases”), such as supplemental oxygen, to a subject. More specifically, this disclosure relates to devices that deliver medical gases to individuals regardless of their orientation (e.g., sleeping position, etc.). Such a device includes a base positionable adjacent to (e.g., around, next to, etc.) an individual's neck and a delivery nozzle, or diffuser, carried by the base to deliver a medical gas to the individual's face (e.g., the individual's nose, the individual's nose and mouth, etc.). Methods for delivering medical gases are also disclosed.

### RELATED ART

A variety of health conditions may lead to the need for the delivery of gases, such as supplemental oxygen. Among other conditions, gases such as supplemental oxygen promote general wellness and helps subjects who suffer from sleep apnea, acute bronchitis, asthma, allergies, heart conditions, chronic obstructive pulmonary disease (COPD), dementia, Alzheimer's disease, and other disorders achieve more stable, restful states of sleep than those subjects typically experience without supplemental oxygen. A variety of apparatuses, including conventional oxygen delivery systems, may be used to provide sleep apnea patients with supplemental oxygen as they sleep.

A conventional oxygen delivery system typically includes an oxygen source (e.g., an oxygen concentrator, an oxygen tank, liquid oxygen, etc.), tubing, and a nasal cannula/oxygen mask that delivers oxygen from the oxygen concentrator to a subject's nose. An oxygen concentrator concentrates oxygen from the air, providing air with an above-ambient concentration of oxygen, which is also referred to herein as “supplemental oxygen” for the sake of simplicity. The oxygen source delivers the supplemental oxygen to and through tubing to the nasal cannula. The nasal cannula has a configuration that enables it to be secured beneath a subject's nose in an orientation that directs the consistent flow of gas, such as supplemental oxygen, into the subject's nose. It is up to the patient to breathe in to access that oxygen from the nasal cannula. If the patient is a mouth breather, as over 70% of people are when they sleep, the concentrated oxygen is not inhaled. Nasal cannulas may also be ineffective for delivering supplemental oxygen to subjects who suffer from upper respiratory tract infections (e.g., a common cold, influenza, sinus infections, etc.) and to subjects who suffer from nasal abnormalities (e.g., a deviated septum, etc.).

The use of a nasal cannula can be uncomfortable and irritating. The presence of the nasal cannula beneath the subject's nose may be bothersome (e.g., it may lead to nose bleeds when the air is dry, such as during wintertime, etc.). In addition, the delivery of supplemental oxygen into a subject's nose has a tendency to dry out the subject's nasal membranes, which may make the nasal cannula even more bothersome. The discomfort a subject experiences while wearing a nasal cannula and while the nasal cannula delivers regulated, supplemental oxygen to the subject's nose may lead to removal of the nasal cannula—either intentionally or unintentionally—as the subject tries to sleep. When the nasal cannula is removed or the subject breathes through their mouth, however, the subject no longer receives supplemental oxygen, which may cause the same problem the supplemental oxygen is intended to address (e.g., fitful, interrupted sleep for hypoxic patients, etc.).

Devices that deliver medical gases to individuals' mouths have been developed. U.S. Pat. No. 11,324,902 describes an example of a pillow that delivers medical gases to individuals as they lie down. While such devices are effective in providing medical gases to individuals who face sideways as they lie down or sleep, their effectiveness diminishes when the individual moves their head to other orientations (e.g., when the individual's head faces upwardly, etc.).

### SUMMARY

Various aspects relating to the delivery of medical gas to an individual are disclosed, including devices, systems, and methods. The devices, systems, and methods of this disclosure include the delivery of medical gas to an individual regardless of the orientation of the individual's head.

A device or system may deliver medical gas to a face of an individual. Such a device may include a base positionable adjacent to (e.g., around, next to, etc.) an individual's neck and a delivery nozzle. The base may carry the delivery nozzle. More specifically, the base may carry the delivery nozzle at a location that will direct the medical gas toward the face of the individual.

The base of the device or system may comprise a neck pillow. The neck pillow may comprise any suitable type of neck pillow/travel pillow known in the art. Without limitation, the neck pillow may comprise a U-shaped cushion or a cushion that wraps around the neck. The neck pillow may include an internal cushion and a cover. In some embodiments, the internal cushion may receive a conduit that conveys medical gas to the delivery nozzle. Such an internal cushion may also include a recess that receives and, optionally, orients at least a portion of the delivery nozzle. The cover of a neck pillow that includes such a cushion may include a conduit opening for the conduit that conveys medical gas to the delivery nozzle and a nozzle opening through which a portion of the delivery nozzle may protrude.

Alternatively, the cover of the neck pillow may accommodate a conduit positionable between the internal cushion of the neck pillow and the cover. Such a cover may include a first conduit opening and a second conduit opening. The first conduit opening may receive an end of the conduit, which may be inserted between the internal cushion and the cover, while the second conduit opening may provide access to the end of the conduit to enable it to be coupled to the delivery nozzle or may enable the end of the conduit to reemerge from the cover so that it can be coupled to the delivery nozzle. The delivery nozzle may be secured to the cover by way of the second conduit opening and/or a fastener on the cover (e.g., a clip, a strap, a fastener (e.g., a touch fastener, etc.) secured to or incorporated into the cover, etc.).

As another alternative, the cover of the neck pillow may include one or more holders (e.g., clips, straps, loops, fasteners, etc.) that receive and hold the conduit in place on an outer surface of the cover, as well as a coupler (e.g., a clip, a loop, a strap, a touch fastener, etc.) that receives and, optionally, orients the delivery nozzle.

The delivery nozzle may comprise any device that can receive, optionally diffuse, and direct medical gas to the individual's face. As an example, the delivery nozzle may comprise a miniaturized version of a diffuser, or “mini-diffuser,” or a standard version of a diffuser. Examples of diffusers include but are not limited to those described by U.S. Patent Application Publication US 2022/0339377 A1 and U.S. Provisional Patent Application No. 63/454,933, the entire disclosures of which are incorporated herein. Such a diffuser couples to a conduit that conveys medical gas from a source to the delivery nozzle and diffuses the medical gas. Such a diffuser may optionally include walls that direct the flow of medical gas to a desired location (e.g., the face of the individual, etc.).

A mini-diffuser may be incorporated into the base. Alternatively, a mini-diffuser may be secured to the base (e.g., to a cover of a neck pillow, etc.) with a suitable coupler (e.g., a clip, a loop, a strap, a fastener, etc.). As another alternative, a mini-diffuser may be secured in place relative to the base while being positionable at a location spaced apart from the base. For example, the mini-diffuser may simply be provided at the end of a conduit (e.g., a tube, etc.) that conveys medical gas from a source to the delivery nozzle. As another example, a positionable arm, or boom, may carry the mini-diffuser and facilitate its positioning relative to an individual's face (e.g., to direct medical gas into or around the individual's mouth, to direct medical gas into the individual's nose, etc.). The boom may couple the mini-diffuser to the base in a manner that facilitates orientation of the mini-diffuser relative to the base and relative to the face of the individual. While in some embodiments the boom may merely comprise an arm that couples the mini-diffuser to the neck pillow and enables positioning of the mini-diffuser, in other embodiments the boom may additionally include a conduit coupled to and effectively extending a conduit coupled to a source of the medical gas to convey the medical gas to the mini-diffuser.

A coupler (e.g., a clip, a loop, a strap, a fastener (e.g., a touch fastener, etc.), etc.) may secure a standard-sized diffuser to the base and, optionally, enable an individual to orient the diffuser relative to the base.

In various embodiments, including but not limited to those where the delivery nozzle comprises a diffuser, the device or system may include a mask. The mask may ensure that medical gas delivered by the delivery nozzle is directed toward the face of the individual and may prolong the amount of time the medical gas is available for inhalation by the individual. The mask may be coupled to the base (e.g., to a cover of a neck pillow, etc.). Alternatively, the mask may be part of the base (e.g., part of the cover of the neck pillow, etc.). Without limitation, the mask may be stowed or stored in a pocket of the base (e.g., in the cover of the neck pillow, etc.). When use of the mask is desired, the mask may be pulled out of or deployed from the pocket and positioned over the delivery nozzle and a portion of the individual's face (e.g., the individual's mouth, the individual's mouth and nose, etc.) to form a tent over the portion of the individual's face. The mask may be deployed and arranged in a manner that helps direct the medical gas from the delivery nozzle to the individual's mouth and/or nose and at least partially contains the medical gas in front of the individual's mouth and/or nose.

A device or system for delivering medical gas may also include a support that orients the delivery nozzle (e.g., diffuser, etc.) at a desired angle relative to the face of the individual.

A method for delivering medical gas to a face of an individual may include positioning a base adjacent to (e.g., around, next to, etc.) a neck of the individual, orienting a delivery nozzle carried by the base toward the face of the individual, and delivering the medical gas to the face of the individual through the delivery nozzle.

Positioning the base may comprise positioning a neck pillow at least partially around the individual's neck.

As the neck pillow is positioned, the delivery nozzle may be oriented relative to the individual's face. Alternatively, orientation of the delivery nozzle may occur after positioning the neck pillow around the individual's neck. Orientation of the delivery nozzle may include orienting the delivery nozzle to direct the medical gas to a mouth or a nose of the individual.

Optionally, a mask may be positioned over a portion of the face of the individual. The mask may be positioned to contain the delivery nozzle, direct medical gas expelled from the delivery nozzle toward the individual's face, and/or hold or contain medical gas in front of the individual's face. The mask may be assembled with the base or it may be deployed from the base (e.g., from a cover of a neck pillow, etc.).

Other aspects of the disclosed subject matter, as well as features and advantages of various aspects of the disclosed subject matter, should be apparent to those of ordinary skill in the art through consideration of the ensuing description, the accompanying drawings, and the appended claims

### BRIEF DESCRIPTION OF THE DRAWINGS

In the dsrawings:

FIG. 1 depicts an embodiment of a neck-supported medical gas delivery system of this disclosure;

FIG. 2 illustrates use of the embodiment of the neck-supported medical gas delivery system of FIG. 1 to deliver medical gas to a subject;

FIG. 3 depicts another embodiment of a neck-supported medical gas delivery system of this disclosure;

FIG. 4 illustrates use of the embodiment of the neck-supported medical gas delivery system of FIG. 3 to deliver medical gas to a subject;

FIG. 5 depicts another embodiment of a neck-supported medical gas delivery system of this disclosure;

FIG. 6 illustrates use of the embodiment of the neck-supported medical gas delivery system of FIG. 5 to deliver medical gas to a subject; and

FIGS. 7-9 are graphs showing the show pulse oximetry (SpO₂), or percent saturation of a subject's blood with oxygen gas (O2), of a subject on various nights while sleeping with the embodiment of the neck-supported medical gas delivery system shown in FIG. 4.

### DETAILED DESCRIPTION

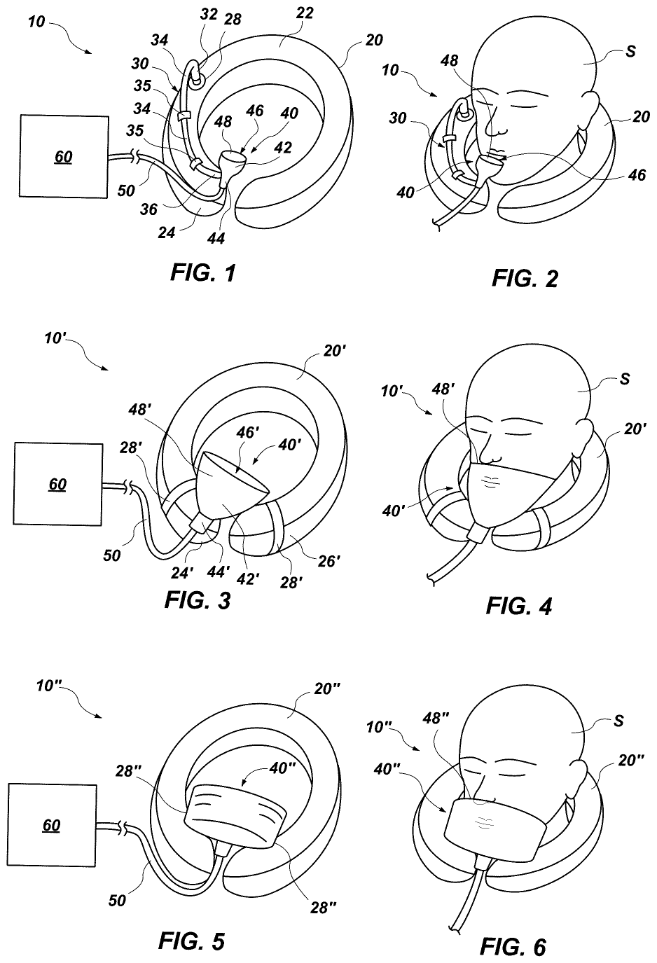

With reference to FIGS. 1 and 2, an embodiment of a neck-supported medical gas delivery system 10, which may be more simply referred to hereinafter as a “gas delivery system 10,” is depicted. The gas delivery system 10 includes a base 20, an optional positioner 30, and a diffuser 40. The base 20 may support the optional positioner 30 and the diffuser 40. The optional positioner 30 may facilitate positioning of the diffuser 40 relative to a face of a subject S. The diffuser 40 may receive a medical gas from a source 60 (e.g., a tank, a generator, etc.) and deliver the medical gas to the subject S's face.

The base 20 of the gas delivery system 10 may be somewhat elongated and flexible to enable a center portion 22 of the base 20 to be wrapped around a back of a neck of the subject S, while ends 24 and 26 may extend over and optionally rest on the subject S's shoulders. In a specific embodiment, the base 20 may have a U shape. In a more specific embodiment, the base 20 include a conformable material, such as a cushion and comprise a so-called “neck pillow.”

The base 20 may carry one or more mounts 28, which may receive the optional positioner 30 and/or the diffuser 40 to secure the positioner 30 and/or the diffuser 40 in place and to orient the diffuser 40 relative to the subject S's face. Each mount 28 may have a configuration that enables it to receive, engage, and orient the positioner 30 and/or the diffuser 40 relative to the subject S's face. Without limitation, each mount 28 may comprise a coupler (e.g., a clip, a loop, a strap, a fastener, etc.) that receives the optional positioner 30 and/or the diffuser 40 and facilitates the removable placement of the diffuser 40 right in front of the subject S's face.

As shown in FIGS. 1 and 2, the mount 28 may be positioned near the center portion 22 of the base 20, such that the mount 28 may be located adjacent to a side of the subject S's neck when the base 20 is placed around the back and sides of the subject S's neck. Such a mount 28 may receive and engage a base 32 of a positioner 30, which may then position and/or orient the diffuser 40.

Alternatively, as shown in FIGS. 3 and 4, one or more mounts 28′ may be positioned near an end 24′, 26′ of the base 20′, such that the mount(s) 28′ may be located near a front of the subject S's face when the base 20′ is placed around the back and sides of the subject S's neck. Such a mount 28′ may receive a diffuser 40′, position the diffuser 40′, and/or orient the diffuser 40′ toward the subject S's face.

With returned reference to FIGS. 1 and 2, in embodiments where the gas delivery system 10 includes the optional positioner 30, the positioner 30 may include a base 32 and a boom 34. The boom 34 may extend from the base 32. An orientation of the boom 34 relative to the base 32 may be adjustable. Without limitation, a joint 33 (e.g., a ball joint, a hinge, etc.) may couple a first end 35 of the boom 34 to the base 32 and enable a second end 36 of the boom 34 to be positioned in a desired location (e.g., in front of the subject S's face, etc.). Alternatively or additionally, the boom 34 itself may be adjustable. More specifically, a shape of the boom 34 may be adjustable. Without limitation, the boom 34 may comprise a material that may enable the boom 34 to be bent into a desired conformation and retain its new conformation, the boom 34 may comprise a plurality of rigid sections that are joined by one or more joints 35 that enable the boom 34 to be bent, the boom 34 may include a combination of these features, or the boom 34 may have any other configuration that enables it to be bent and to retain its adjusted shape.

The second end 36 of the boom 34 may couple to the diffuser 40. As depicted by FIGS. 1 and 2, a diffuser 40 that is carried by a positioner 30 may be relatively small and may be designed to be spaced apart from the subject S's face and to direct medical gas toward a desired location of the subject S's face, such as the subject S's mouth and/or nose.

The diffuser 40 includes a body 42 with an inlet 44, a cavity 46, and a delivery face 48. The body 42 may increase in size (e.g., taper outwardly, etc.) from the inlet 44 to the delivery face 48, which may be located at an opposite end of the body 42 from the inlet 44. The inlet 44 may be coupled to a gas delivery tube 50 that extends from and communicates with the source 60.

An interior of the body 42 defines the cavity 46, which has a shape and, optionally, one or more features that define a manner in which gas flows from the inlet 44 to the delivery face 48. The gas may flow out of the diffuser 40 through the delivery face 48. As depicted, the cavity 46 may be somewhat enlarged relative to the gas delivery tube 50 and the inlet 44 to decrease a flow rate of gas as it exits the inlet 44, flows through the cavity 46, and flows from the delivery face 48, while enabling the gas to continue to flow at a sufficient rate to reach the subject S's face without significant losses from diffusing in the gap or space between the delivery face 48 and the subject S's face.

Referring again to FIGS. 3 and 4, a gas delivery system 10′ with a larger embodiment of diffuser 40′ is depicted. The diffuser 40′ may have a configuration that enables it to be positioned over a portion of a subject S's face so as to deliver gas directly to the subject S's face (e.g., their nose and/or mouth). The diffuser 40′ may be secured directly to a base 20′ of the gas delivery system 10′. Optionally, the diffuser 40′ may be attached to the base 20′ at any of a variety of different locations to accommodate individuals with different sizes of heads.

The diffuser 40′ includes a body 42′ with an inlet 44′, a cavity 46′ and a delivery face 48′. The body 42′ may increase in size (e.g., taper outwardly, etc.) from the inlet 44′ to the delivery face 48′, which may be located at an opposite end of the body 42′from the inlet 44′. The inlet 44′ may be coupled to a gas delivery tube 50 that extends from and communicates with the source 60.

An interior of the body 42′ defines the cavity 46′, which has a shape and, optionally, one or more features that define a manner in which gas flows from the inlet 44′ to the delivery face 48′. As depicted, a shape of the cavity 46′ may enable it to decrease a flow rate of gas as it exits the inlet 44′, flows into and through the cavity 46′, and flows through the delivery face 48′. The shape of the cavity 46′ may enable it to retain a predetermined volume of the gas, which is to be inhaled by the subject S.

A configuration of the delivery face 48′ may enable it to receive the portion of the subject S's face over which the diffuser 40′ is positioned (e.g., their nose and/or mouth), to form a tent that holds gas over the subject S's face, and to rest comfortably against the subject S's face. Thus, the diffuser 40′ may comprise a mask. In some embodiments, the delivery face 48′ may rest against the subject S's face without sealing against the subject S's face. In other embodiments, the delivery face 48′ may seal against the subject S's face. Notably, the gas delivery system 10′ lacks a positioner 30 (FIGS. 1 and 2); the diffuser 40′ may be coupled directly to the base 20′.

As an alternative to mounting the diffuser 40′ to the base 20′, as shown in FIGS. 3 and 4, a gas delivery system 10″ may include a diffuser 40″ that is integrated into its associated base 20″, as shown in FIGS. 5 and 6. The base 20″ of such gas delivery system 10″ may include a receptacle 28″ that receives a portion of the diffuser 40″. The receptacle 28″ may be located adjacent to or in a neck-facing surface of the base 20″. The receptacle 28″ may orient the delivery face 48″ of the diffuser 40″ upward toward a location where the subject S's face will be positioned as the subject S wears the gas delivery system 10″. The receptacle 28″ may comprise a recess in an internal member (e.g., a cushion, etc.) of the base 20″ and an opening through an outer surface (e.g., a cover, etc.) of the base 20″.

U.S. Patent Application Publication US 2022/0339377 A1 (“the '377 Publication”) and International Application No. PCT/US 2024/021801, filed on Mar. 27, 2024 and titled MEDICAL GAS DELIVERY (“the '801 International Application”), provide further detail regarding various embodiments of diffusers 40, 40′, 40″ that may be used to deliver a medical gas to the face of a subject S. The entire disclosures of the '377 Publication and the '801 International Application are hereby incorporated herein.

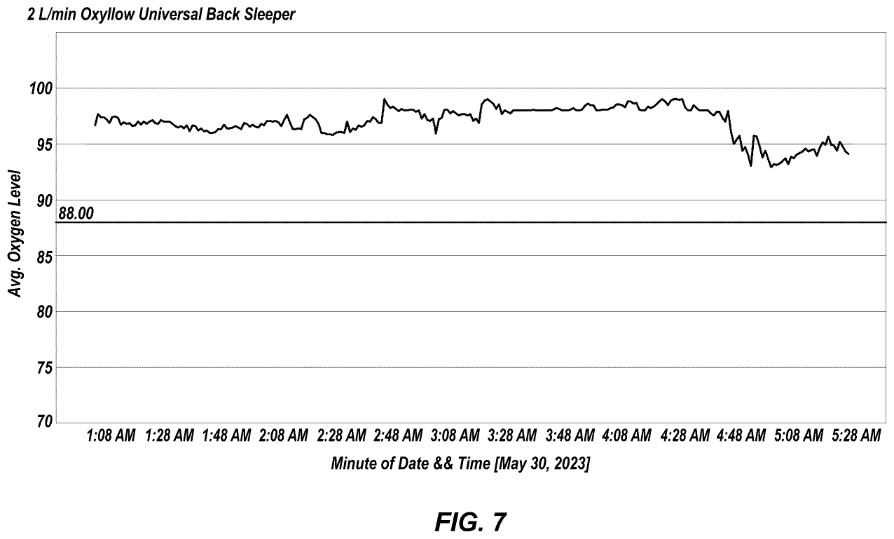

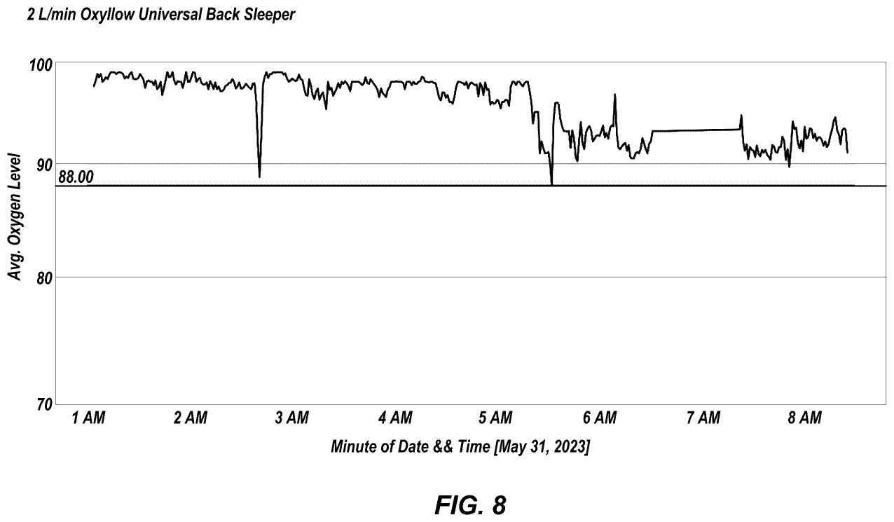

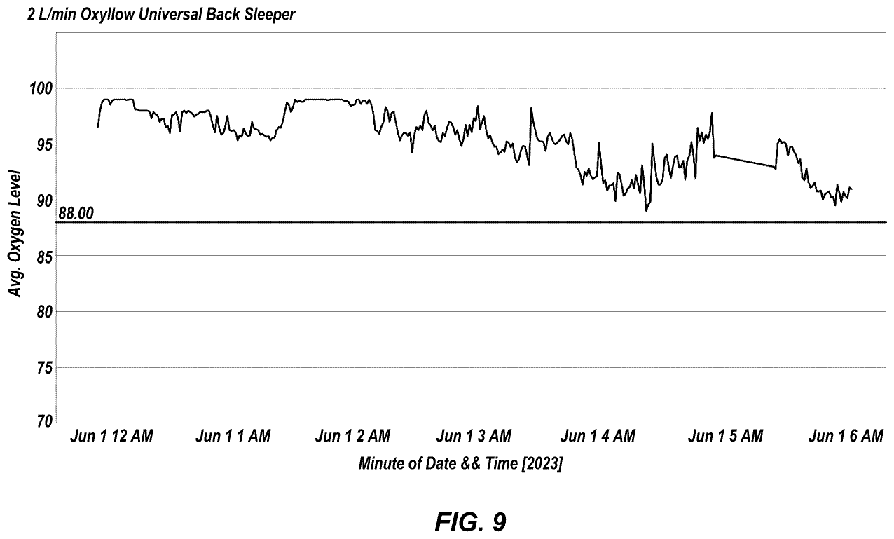

Turning now to FIGS. 7-9, graphs are depicted that show SpO₂ measurements of a subject who slept with the embodiment of gas delivery system 10′ shown in FIGS. 3 and 4 on three consecutive nights. Without supplemental oxygen, the subject's SpO₂ dropped to inadequate levels (i.e., below 88%). The individual typically slept on their back, with their face oriented upwardly. The base 20′ was secured around the subject's neck and supplemental oxygen was delivered to the subject through the diffuser 40′. SpO₂ measurements were obtained continuously throughout the night.

As can be seen, the gas delivery system 10′ delivered sufficient supplemental oxygen to enable the subject to consistently maintain adequate SpO₂ (over 88%) all night on each of the three nights the test was conducted.

The disclosed devices and systems enable individuals to receive medical gases regardless of the orientations of their heads.

Although this disclosure and the accompanying drawings provide many specifics, they should not be construed as limiting the scopes of any of the appended claims, but merely as providing information pertinent to some specific embodiments that may fall within the scopes of the appended claims. Features from different embodiments may be employed in combination. In addition, the scopes of the appended claims may encompass other embodiments. All additions to, deletions from, and modifications of the disclosed subject matter that fall within the scopes of the claims are to be embraced by the claims.

What is claimed:

1. A device that delivers gas or medical gas to a face of an individual, comprising:
a neck pillow positionable around a neck of the individual; and
a delivery nozzle carried by the neck pillow at a location that will direct the medical gas toward the face of the individual.

2. The device of claim 1, wherein the delivery nozzle comprises a mini-diffuser.

3. The device of claim 2, further comprising:
a boom that orients the mini-diffuser relative to the face of the individual.

4. The device of claim 3, wherein the boom comprises a conduit that conveys the medical gas to the mini-diffuser.

5. The device of claim 1, wherein the delivery nozzle comprises a diffuser.

6. The device of claim 5, further comprising:
a mask associated with the neck pillow and positionable over a portion of the face of the individual.

7. The device of claim 6, wherein the mask is positionable over a mouth of the individual.

8. The device of claim 7, wherein the mask is further positionable over a nose of the individual.

9. The device of claim 6, wherein the mask comprises an extension of a cover of the neck pillow.

10. The device of claim 9, wherein the mask is stowable within a pocket of the cover of the neck pillow.

11. The device of claim 6, wherein the mask is deployable to form a tent over the portion of the face of the individual.

12. The device of claim 5, further comprising:
a support that orients the diffuser at a desired angle relative to the face of the individual.

13. The device of claim 1, wherein the delivery nozzle is incorporated into the neck pillow.

14. The device of claim 1, further comprising:
a conduit that conveys the medical gas from a source toward the delivery nozzle.

15. A method for delivering medical gas to a face of an individual, comprising:
positioning a neck pillow around a neck of the individual;
orienting a delivery nozzle carried by the neck pillow toward the face of the individual; and
delivering the medical gas to the face of the individual through the delivery nozzle.

16. The method of claim 15, wherein positioning the neck pillow includes orienting the delivery nozzle.

17. The method of claim 15, wherein orienting the delivery nozzle occurs after positioning the neck pillow.

18. The method of claim 17, wherein orienting the delivery nozzle comprises orienting the delivery nozzle to direct the medical gas to a mouth or a nose of the individual.

19. The method of claim 15, further comprising:
positioning a mask over a portion of the face of the individual, the mask containing the delivery nozzle.

20. The method of claim 19, wherein positioning the mask comprises deploying the mask from the neck pillow.
